(19) United States
(12) Patent Application Publication
(10) Pub. No.: US 2004/0234969 A1
(43) Pub. Date: Nov. 25, 2004

(54) Mehtod for examining steroid-responsiveness

(76) Inventors: Sugita Yuji, Ibaraki (JP); Masayuki Heishi, Konohana-ku, Osaka (JP); Shinji Kagaya, Tokyo (JP); Shigemichi Gunji, Tokyo (JP); Hiroshisa Saito, Tokyo (JP)

Correspondence Address: Peter G Caroll, Medlen & Carroll, Suite 350, 101 Howard Street, San Franciso, CA, 94105, US

(21) Appl. No.: 10/469,489

(22) PCT Filed: Mar. 1, 2002
(86) PCT No.: PCT/JP02/01915

(30) Foreign Application Priority Data
Mar. 1, 2001 (JP) 2001-56432

(51) Int. Cl. C12Q 1/68

(52) U.S. Cl. 435/6

(57) ABSTRACT

CYP1B1 was obtained as a gene whose expression levels in mononuclear cells greatly differs between a steroid responder group and a poorly steroid responder group of atopic dermatitis patients. CYP1B1 is a gene whose expression levels are reduced in mononuclear cells of the poorly steroid responder group. The present invention provides a method for testing steroid responsiveness and a method of screening for a compound that is useful in improving steroid responsiveness using the expression level of this gene in biological samples as an indicator.

### TECHNICAL FIELD

The present invention relates to a method for testing steroid responsiveness.

### BACKGROUND ART

Allergic diseases such as atopic dermatitis are considered multifactorial diseases. These diseases are caused by the interaction of many different genes, whose expressions are influenced by multiple diverse environmental factors. Thus, determination of specific genes causing a specific disease has been extremely difficult for allergic diseases.

Additionally, expression of mutated or defective genes, or over expression or reduced expression of specific genes is thought to be involved in allergic diseases. To elucidate the role of gene expression in diseases, it is necessary to understand how a gene is involved in triggering the onset of a disease and how the expression of the gene is altered by external stimulants such as drugs.

To date, administration of steroid drugs has become one of the most common treatment for allergic diseases. For example, external steroid preparations are considered to be effective for atopic dermatitis. Furthermore, inhalation or oral administration of steroid preparations is regarded as one of the important treatments for bronchial asthma. Steroid preparations suppress the production of inflammatory cytokines and activity of activated eosinophils through stimulation of glucocorticoid receptors (GR). As a result, steroids are considered to relieve inflammatory symptoms causing therapeutic effects on allergic diseases.

Indeed, steroids have become an important means to treat allergic diseases; however, certain inflammatory symptoms persist for which steroids are less responsive. When no therapeutic effect by steroids can be observed, it is referred to as “steroid-resistant”. Furthermore, patients are classified according to a clinical score (a modified Leicester score) of responsiveness toward steroid ointment treatment: “responder” when the clinical score is improved ⅓ or more of the original value after two weeks from the treatment, and “poor-responder” when the improvement in the score is less than ⅓. Various causes are likely to be involved in the resistance and poor response to steroids.

First, obviously no therapeutic effect can be expected by administering steroids for pathophysiologies involving a pathway that cannot be controlled with steroids. Such cases are inadaptable for steroid drugs, and thus steroids should not be administered to such diseases. Steroid responsiveness becomes an important issue in cases where no treatment effect can be obtained for a steroid due to the patient's diathesis in spite of the originally guaranteed effectiveness of the steroid on a particular disease.

For a poor steroid responder, a treatment other than that with steroids must be selected. While administering steroids, one has to consider side effects like adrenal cortex dysfunction and eyesight impediment such as cataract and glaucoma. Furthermore, side effects such as dermatrophy, steroid purpura and steroid dermatitis are sometimes observed by topical administration of steroids. Therefore, exposure of patients to critical side effects by ineffective steroid administration should be avoided. To select a safer treatment method for a patient, it is ideal to predict steroid responsiveness of the patient prior to steroid administration. Furthermore, regardless of the presence or absence of steroid side effects, it is essential to select a treatment that is most effective for patient. However, to date, no diagnostic technique enabling prediction of steroid responsiveness is disclosed in the art. Therefore, without the actual administration of steroids, poor steroid responsiveness of a patient cannot be observed.

The cause of steroid-resistance has not been fully elucidated. For example, aberration in the post-translational modification of glucocorticoid receptors (GR), a target of steroids, has been indicated as a possible cause of steroid-resistance (Picard, D. Nature 348:166-168, 1990, “Reduced levels of hsp90 compromise steroid receptor action in vivo.”). Alternatively, in the case wherein numerous inflammatory transcription factors are associated with inflammation, controlling all these factors is assumed to exceed the limit of the controlling ability of steroids, thereby resulting in steroid-resistance. Furthermore, as the mechanism of steroid-resistance, CBP (CREB-binding protein), a transcriptional coactivator of gene transcription, are suggested to be consumed by transcriptional activation of other genes, and genes that are essential for the immunosuppression by steroids may only insufficiently transcribed (Kamei, Y. et al. Cell 85: 403-414, 1996, “A CBP integrator complex mediates transcriptional activation and AP-1 inhibition by nuclear receptors”). However, none of these reports sufficiently explains the mechanism of poor steroid responsiveness. Prediction of poor steroid responsiveness requires elucidation of the cause thereof.

Elucidation of the cause of poor steroid responsiveness enables not only prediction of steroid responsiveness but also provides novel therapeutic methods. For example, discovery of a causative molecule causing reduced steroid responsiveness enables methods to raise steroid responsiveness via functional inhibition of the molecule, thereby and promoting the therapeutic effect of steroids. Alternatively, when the cause of reduced steroid responsiveness is quantitative shortage of a specific molecule, steroid responsiveness is expected to be improved by supplementary administration of that molecule.

A variety of treatment methods have been attempted for allergic diseases. However, steroid use is still an important option of therapies for allergic diseases. Even to date, no drug other than steroids exists that exerts excellent therapeutic effects on a wide variety of disorders. Therefore, the realization of effective steroid treatments against patient with poor steroid responsiveness will be welcome news for such patients.

In addition, transition to secondary hyperparathyroidism in kidney dialysis patients during activated vitamin D₃ treatment can be mentioned as a pathophysiology caused by insufficient therapeutic effects of steroids. The activated vitamin D₃ is a typical steroid that controls the function of parathyroid. However, due to the insufficient action of the activated vitamin D₃ in patients with poor steroid responsiveness, a transition to secondary hyperparathyroidism has been observed.

Therefore, elucidation of the cause of changes in steroid responsiveness is highly significant.

### DISCLOSURE OF THE INVENTION

An object of the present invention is to provide a gene that serves as an indicator for steroid responsiveness. Furthermore, another object of the present invention is to provide a method for testing steroid responsiveness and a method of screening for a compound to raise steroid responsiveness based on the indicator.

The present inventors considered that elucidation of genes associated with steroid responsiveness would be useful for diagnosis and treatment of steroid responsiveness. Therefore, the inventors searched for genes whose expression levels differed between patients who responded to steroid treatment and those who only poorly respond thereto. The use of DNA chips is advantageous to observe differences in expression levels of numerous genes among cells under a specific condition. To search for target genes among a wide range of genes, the present inventors used a DNA chip that enables analysis of approximately 5,600 kinds of genes. Furthermore, to discover specific genes with an expression level that changes in association with steroid responsiveness and poor responsiveness of subjects, the inventors selected genes with a change in the expression level of 3-fold or more between responsive and poorly responsive subjects.

Then, the expression level of the genes obtained by searching was analyzed in a plurality of atopic dermatitis patients. As a result, the inventors succeeded in isolating a gene, CYP1B1, whose expression level was significantly reduced in patients with poor steroid responsiveness as compared to patients responding to steroid therapy. Furthermore, the inventors found that steroid responsiveness can be tested and compounds to raise steroid responsiveness can be screened using this gene as an indicator and completed this invention. Specifically, the present invention relates to a method for testing steroid responsiveness as well as a method of screening for a compound to raise steroid responsiveness as described below:

[1] a method for testing steroid responsiveness, comprising the steps of:

a) measuring the expression level of the CYP1B1 gene in a biological sample of a subject; and

b) comparing the measured expression level to that of the gene in a biological sample from a steroid responsive subject;

[2] the method according to [1], wherein steroid responsiveness in allergic diseases is tested;

[3] the method according to [2], wherein the allergic disease is atopic dermatitis;

[4] the method according to [1], wherein the expression level of the gene is measured by PCR of cDNA.;

[5] the method according to [1], wherein the expression level of the gene is measured by detecting protein encoded by the gene;

[6] a reagent for testing steroid responsiveness, said reagent comprising an oligonucleotide having a nucleotide sequence complementary to a polynucleotide comprising the nucleotide sequence of the CYP1B1 gene or to the complementary strand thereof, which oligonucleotide has a length of at least 15 nucleotides;

[7] a reagent for testing steroid responsiveness, said reagent comprising an antibody recognizing peptides containing the amino acid sequence of the CYP1B1 protein;

[8] a method of screening for a compound that raises steroid responsiveness, comprising the steps of:

(1) contacting a candidate compound with a cell that expresses the CYP1B1 gene and/or a gene functionally equivalent thereto;

(2) measuring the expression level of the gene; and

(3) selecting a compound that elevates the expression level of the gene compared to that in a control cell that has not been contacted with the candidate compound;

[9] the method according to [8], wherein the cell is a mononuclear cell line;

[10] a method of screening for a compound that raises steroid responsiveness, comprising the steps of:

(1) administering a candidate compound to a test animal;

(2) measuring the expression intensity of the CYP1B1 gene and/or a gene functionally equivalent thereto in a biological sample from the test animal; and

(3) selecting a compound that elevates the expression level of the gene compared to that of a control animal without administration of the candidate compound;

[11] a method of screening for a compound that raises steroid responsiveness, comprising the steps of:

(1) contacting a candidate compound with a cell transfected with a vector comprising the transcriptional regulatory region of the CYP1B1 gene and/or a gene functionally equivalent thereto, and a reporter gene that is expressed under the control of the transcriptional regulatory region;

(2) measuring the activity of the reporter gene; and

(3) selecting a compound that elevates the expression level of the gene compared to that of a control cell that has not been contacted with the candidate compound;

[12] a method of screening for a compound that raises steroid responsiveness, comprising the steps of:

(1) contacting a candidate compound with the CYP1B1 protein and/or a protein functionally equivalent thereto;

(2) measuring the activity of the protein; and

(3) selecting a compound that elevates the activity of the protein compared to that of the control protein that has not been contacted with the candidate compound;

[13] a pharmaceutical to raise steroid responsiveness, which comprises a compound that can be obtained by the method according to any one of [8], [10], [11] and [12] as an effective ingredient;

[14] a pharmaceutical to raise steroid responsiveness, which comprises the CYP1B1 gene or the CYP1B1 protein as the main ingredient;

[15] a therapeutic agent for poor steroid responsive disorders comprising the pharmaceutical to raise steroid responsiveness according to [13] and/or [14] in combination with a steroid drug;

[16] a kit for screening a candidate compound for a therapeutic agent to raise steroid responsiveness, said kit comprising a polynucleotide containing at least 15 nucleotides wherein the polynucleotide is complementary to a polynucleotide comprising the nucleotide sequence of the CYP1B1 gene or the complementary strand thereof, and a cell expressing the CYP1B1 gene;

[17] a kit for screening a candidate compound for a therapeutic agent to raise steroid responsiveness, said kit comprising an antibody recognizing a peptide containing the amino acid sequence of the CYP1B1 protein, and a cell expressing the CYP1B1 gene; and

[18] the use of a transgenic non-human vertebrate, whose expression intensity of the CYP1B1 gene and/or a gene functionally equivalent thereto in mononuclear cells is decreased, as a model animal for poor steroid responsiveness.

The present invention also relates to a method for improving steroid responsiveness comprising the step of administering a compound that can be obtained by the screening method according to any one of the aforementioned [8], [10], [11] and [12]. The present invention further relates to the use of the compounds which can be obtained by the screening method according to any one of the above-described [8], [10], [11] and [12] in the preparation of pharmaceuticals to raise steroid responsiveness. Furthermore, the present invention relates to a method for improving steroid responsiveness comprising the step of administering the CYP1B1 gene or the CYP1B1 protein. Moreover, this invention relates to the use of the CYP1B1 gene or CYP1B1 protein in the preparation of pharmaceuticals to raise steroid responsiveness.

The CYP1B1 gene is a gene whose existence had been demonstrated. This gene encodes one of the molecular species of cytochrome P450, whose expression is induced by treating cultured cells with dioxins. While the CYP1B1 gene is expressed in a large number of tissues, including eye tissue, heart, brain, lung, liver, skeletal muscle, kidney and pancreas, specific expression in monocytes and macrophages among blood cells is observed (Baron, J. M., Zwadlo-Klarwasser, G., Jugert, F., Hamann, W., Rubben, A., Mukhtar, H., Merk, H. F., Biochem. Pharmacol. 56: 1105-10, 1998, “Cytochrome P450 1B1: a major P450 isoenzyme in human blood monocytes and macrophage subsets.”). Among the revealed association of the CYP1B1 gene with various disorders, a point mutation of the gene has been reported to be involved in congenital glaucoma (http://www.ncbi.nlm.nih.gov/htbin-post/Omim/dispmim?601771). However, no relation of the gene to steroid responsiveness has been reported so far.

Herein, “steroid responsiveness” refers to the magnitude of the therapeutic effect of a steroid on allergic reactions or inflammatory symptoms that is achieved following its administration. Steroid responsiveness is not only assessed for allergic disorders but also for all kind of diseases for which a steroid treatment has been considered effective. Patients whose symptoms ameliorate by steroid administration are steroid-responsive. In contrast, the state with no therapeutic effect by a steroid is referred to as steroid-resistant, and those with only slight effect are referred to as poor steroid responsive.

The steroid efficacy on allergic disorders can be quantitatively assessed by comparing the diagnostic indicator of allergic symptoms. For example, for atopic dermatitis, a typical allergic disorder, the atopic dermatitis/clinical score system has been known (Leicester system, Sowden, J. M. et al., Lancet, 338: 137-40, 1991, “Double-blind controlled crossover study of cyclosporin in adults with severe refractory atopic dermatitis.”). According to the method, the symptoms of dermatitis are numerically expressed based on the progress and developmental location of dermatitis. In addition, the number of peripheral blood eosinophils can be used as an indicator of symptoms of allergic disorders. The therapeutic effects of a steroid can be assessed by comparing these indicators before and after the administration of the steroid.

In atopic dermatitis, using the clinical score (modified Leicester score) of the responsiveness to steroid ointment treatment, patients whose score value is improved by ⅓ or more after two weeks from the initiation of the treatment are categorized as a “responder”, and patients with an improvement smaller than ⅓ are categorized as a “poor-responder”. For disorders other than atopic dermatitis, patients can be ranked according to their steroid-responsiveness using an assessment scale of therapeutic effect adapted for each disorder.

Herein, the term “allergic disease” is a general term for diseases in which allergic reaction is involved. More specifically, it is defined as a disease in which an allergen must be identified, a strong correlation between the exposure to the allergen and the onset of the pathological change must be demonstrated, and the pathological change must be proven to have an immunological mechanism. Herein, an immunological mechanism means that immune responses by the leukocytes are induced by the stimulation of the allergen. Examples of allergens include mite antigen and pollen antigen.

Representative allergic diseases include atopic dermatitis, allergic rhinitis, pollen allergy and insect allergy. Allergic diathesis is a genetic factor that is inherited from allergic parents to their children. Familial allergic diseases are also called atopic diseases, and the causative factor that is inherited is the atopic diathesis. The term “asthma” is a general term for atopic diseases with respiratory symptoms among atopic diseases.

A method for testing steroid responsiveness according to the present invention comprises the steps of (1) measuring the expression level of the CYP1B1 gene in a biological sample of a subject, and (2) comparing the measured value with that of a steroid-responsive patient. As a result of comparison between the two values, when the expression level of said gene in the subject is significantly reduced compared to that in the steroid-responsive patient, the subject is judged a poor-responder to steroids. Herein, the CYP1B1 gene that serves as an indicator for steroid responsiveness is also simply referred to as the “indicator gene”. According to the present invention, the CYP1B1 gene includes homologues not only from human but also from other species. Therefore, an indicator gene for species other than human, unless otherwise indicated, refers to either an intrinsic CYP1B1 gene homologue of that particular species or an extraneous CYP1B1 gene transformed into the body of the particular species.

In this invention, a homologue of the human CYP1B1 gene refers to a gene derived from species other than human and which hybridizes under stringent conditions to the human CYP1B1 gene used as a probe. Stringent conditions generally include conditions such as hybridization in 4× SSC at 65° C. followed by washing with 0.1× SSC at 65° C. for 1 h. Temperature conditions for hybridization and washing that greatly influence stringency can be adjusted according to the melting temperature (Tm). The Tm changes with the ratio of constitutive nucleotides in the hybridizing base pairs and the composition of hybridization solution (concentrations of salts, formamide and sodium dodecyl sulfate). Therefore, considering these conditions, those skilled in the art can empirically select appropriate conditions to achieve a stringency equal to the condition described above.

Herein, the expression level of an indicator gene includes transcription of the gene to mRNA as well as translation into protein. Therefore, the method for testing steroid responsiveness according to the present invention is performed based on the comparison of the expression intensity of mRNA corresponding to the aforementioned indicator gene or the expression level of a protein encoded by the gene.

For comparing the expression levels, usually a standard value is set based on the expression level of the above-described indicator gene in a steroid responder group. Based on this standard value, a permissible range is set, for example, at ±2 S.D. Methods for setting the standard value and permissible range based on the measured values of the indicator gene is well known in the art. When the expression level of the indicator gene in a subject is lower than the permissible range, the subject is predicted to be a poor steroid responder.

Measurement of the expression level of the indicator gene in the testing for steroid responsiveness according to the present invention can be performed according to gene analytical methods known in the art. More specifically, for example, the hybridization technique using a nucleic acid hybridizing to the indicator gene as a probe, and gene amplification technique using a DNA hybridizing to the gene of this invention as a primer can be utilized for the measurement.

Probes and primers used in the testing according to this invention can be designed based on the nucleotide sequence of the above-described indicator gene. The nucleotide sequence of the indicator gene is known with a GenBank accession No. U03688. The nucleotide sequence of CYP1B1 gene is also set forth in SEQ ID NO: 11, and the amino acid sequence encoded by the nucleotide sequence in SEQ ID NO: 12.

Furthermore, generally, genes of higher animals are, with high frequency, accompanied by polymorphism. Moreover, many molecules exist for which isoforms consisting of different amino acid sequences are produced during the splicing process. Genes containing mutations in the nucleotide sequence due to polymorphisms or isoforms are also included in the indicator gene of the present invention so long as they have a similar activity to the above-described indicator gene and are associated with steroid responsiveness.

As a primer or probe for the test according to the present invention, a polynucleotide of at least 15 nucleotides and that are complementary to the polynucleotide comprising the nucleotide sequence of the indicator gene or the complementary strand thereof can be utilized. Herein, the term “complementary strand” means one strand of a double stranded DNA composed of A:T (U for RNA) and G:C base pairs to the other strand. In addition, “complementary” means not only those completely complementary to a region of at least 15 continuous nucleotides, but also having a homology of at least 70%, preferably at least 80%, more preferably 90%, and even more preferably 95% or higher. The degree of homology between nucleotide sequences can be determined by the algorithm such as BLAST.

Such polynucleotides are useful as probes to detect the indicator gene, or as primers to amplify the indicator gene. When used as a primer, those polynucleotides comprise usually 15 bp to 100 bp, preferably 15 bp to 35 bp of nucleotides. When used as a probe, DNAs comprising the whole sequence of the indicator gene, or a partial sequence thereof (or its complementary strand) that contains at least 15-bp nucleotides can be used. When used as a primer, the 3′ region thereof must be complementary to the indicator gene, while restriction enzyme-recognition sequences or tag may be linked to the 5′site.

The “polynucleotides” of the present invention may be either DNA or RNA. These polynucleotides may be either synthetic or naturally occurring. In addition, DNA used as a probe for hybridization is usually labeled. Examples of labeling methods are those as described below. Herein, the term “oligonucleotide” means a polynucleotide with relatively low degree of polymerization. Oligonucleotides are included in polynucleotides.

nick translation labeling using DNA polymerase I;

end labeling using polynucleotide kinase;

fill-in end labeling using Klenow fragment (Berger, S L, Kimmel, A R. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. Nucleic Acid Res., 12: 7035-7056, 1984); and

non-isotopic labeling of DNA by incorporating modified nucleotides (Kricka, L J. (1992) Nonisotopic DNA Probing Techniques. Academic Press).

For testing steroid responsiveness using hybridization techniques, for example, Northern hybridization, dot blot hybridization or DNA chip technique may be used. Furthermore, gene amplification techniques, such as RT-PCR method may be used. By using the PCR amplification monitoring method during the gene amplification step in RT-PCR, one can achieve a more quantitative analysis for the gene expression in the present invention.

In the PCR gene amplification monitoring method, the detection target (DNA or reverse transcript of RNA) is hybridized to probes that are dual-labeled at both ends with different fluorescent dyes whose fluorescence cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al., Proc. Natl. Acad. Sci. USA 88: 7276-7280, 1991; Livak, K. J. et al., PCR Methods and Applications 4(6): 357-362, 1995; Heid, C. A. et al., Genome Research 6: 986-994, 1996; Gibson, E. M. U. et al., Genome Research 6: 995-1001, 1996). For the PCR amplification monitoring method, for example, ABI PRISM7700 (Applied Biosystems) may be used.

The method of testing steroid responsiveness of the present invention can also be carried out by detecting a protein encoded by the indicator gene. Hereinafter, a protein encoded by the indicator gene is referred to as an indicator protein. Such test methods are, for example, those utilizing antibodies binding to an indicator protein, including the Western blotting method, the immunoprecipitation method and the ELISA method.

Antibodies that bind to the indicator protein used in the detection may be produced by techniques known to those skilled in the art. Antibodies used in the present invention may be polyclonal or monoclonal antibodies (Milstein, C. et al., Nature 305 (5934): 537-40, 1983). For example, polyclonal antibodies against the indicator protein may be produced by collecting blood from mammals sensitized with an antigen and separating the serum from this blood using known methods. As polyclonal antibodies, the serum containing polyclonal antibodies may be used. According to needs, a fraction containing polyclonal antibodies can be further isolated from this serum. Alternatively, a monoclonal antibody can be obtained by isolating immune cells from mammals sensitized with an antigen; fusing these cells with myeloma cells and such; cloning hybridomas thus obtained; and collecting the antibody from the culture as the monoclonal antibody.

To detect the indicator protein, these antibodies may be appropriately labeled. Alternatively, instead of labeling the antibodies, a substance that specifically binds to antibodies, for example, protein A or protein G, may be labeled to arrange an indirect detection of the proteins. More specifically, one example of an indirect detection method is ELISA.

A protein or partial peptides thereof that is used as an antigen may be obtained, for example, by inserting a gene or portion thereof into an expression vector, introducing it into an appropriate host cell to produce a transformant, culturing the transformant to express the recombinant protein, and purifying the expressed recombinant protein from the culture or the culture supernatant. Alternatively, oligopeptides consisting of the amino acid sequence encoded by the gene or partial amino acid sequences of the amino acid sequence encoded by the full-length cDNA are chemically synthesized to be used as the antigen.

Furthermore, according to the present invention, the testing for steroid responsiveness can be conducted using not only the expression level of the indicator gene but also the activity of the indicator protein in a biological sample as an indicator. The activity of the indicator protein refers to the biological activity inherent in each protein.

CYP1B1, the indicator protein of the present invention, is an enzyme that metabolizes chemical substances such as PCB. Therefore, its activity can be detected using these chemicals as the substrate to measure the metabolic product thereof. The measurement of the metabolic product can be performed using HPLC and the like.

In the testing method of this invention, usually biological samples of subjects are used as the test specimen. Although, blood, sputum, tunica mucosa nasi secretion etc. may be used as the biological sample, it is preferable to use peripheral blood mononuclear cells. The method of collecting mononuclear cells from peripheral blood and such is known in the art. Mononuclear cells isolated, in particular, from peripheral blood are referred to as peripheral blood mononuclear cell (PBMC). Mononuclear cells can be easily collected from, for example, heparinized blood by the specific gravity centrifugation method. Mononuclear cells are a cell population containing monocytes and lymphocytes. The use of mononuclear cells present in a large quantity in peripheral blood facilitates the collection of test samples. Thus, a simple bedside test becomes possible. Lysate prepared by fragmenting the isolated mononuclear cells can be used as a specimen for immunological measurement of the above-described protein. Alternatively, mRNA extracted from this lysate may be used as a specimen for the measurement of mRNA corresponding to the aforementioned indicator gene. The extraction of lysate and mRNA from mononuclear cells can be conveniently carried out using commercial kits. Moreover, when the indicator protein is secreted into the blood stream, the amount of this target protein contained in a humor sample such as blood and serum of subjects may be measured to enable comparison of the expression levels of the gene encoding said protein. According to needs, the aforementioned specimens can be used in the method of this invention after being diluted with a buffer and the like.

In the case of measuring mRNA, the measured value of the CYP1B1 gene expression level in the present invention can be corrected by known methods. The correction enables comparison of changes in the expression levels of the gene in cells. According to this invention, based on the measured value of the expression level of a gene (for example, housekeeping gene) whose expression level in each cell in the above-described biological samples does not widely fluctuate, the measured values of the expression levels of the CYP1B1 gene are corrected. Examples of genes whose expression levels do not widely fluctuate include those encoding β-actin and GAPDH.

Test for steroid responsiveness in the present invention includes the following. Specifically, when steroid treatment is applied to a patient showing atopic dermatitis symptoms, steroid responsiveness of the patient can be predicted based on the present invention prior to the administration of steroids. More specifically, the decrease in the expression level of the indicator gene in a patient indicates a high possibility that the patient is a poor-responder to steroid, and a treatment other than steroid therapy is recommended.

Steroid administration is accompanied by the risk of side effects as described above. Furthermore, prediction of therapeutic effects prior to the initiation of treatment leads to immediate relief of patient from agony to improve his/her QOL. Therefore, the testing method of the present invention provides extremely important information on the selection of therapeutic plans for allergic diseases.

Alternatively, a gene whose expression level changes in response to steroid can be expected to be useful as an indicator for the decrease of type 1 helper T cells (Th1 cells). The decrease of Th1 cell function in comparison to the type 2 helper T cells (Th2 cells) is considered as one of the causes of allergic diseases. According to this concept, allergic symptoms are caused because of relative enhancement of the function of Th2 cells inducing IgE antibody production to Th1 cells. The increase in the number of Th2 cells and decrease of Th1 cells may be the cause of the relative decrease of the function of Th1.

Patients with atopic dermatitis (AD) with decreased IFN-γproductivity have been reported to have increased levels of IgE antibody specific to Candida (Kimura, M., Tsuruta, S., Yoshida, T., Int. Arch. Allergy Immunol. 122: 195, 2000, “IFN-gamma plays a dominant role in upregulation of Candida-specific IgE synthesis in patients with atopic dermatitis.”). IFN-γ is a typical Th1 cytokine. Thus, patients with AD due to the decrease in Th1 cells have decreased resistance to fungi and viruses and thus resident Candida is likely to be increased. As a result, the IgE level against Candida may be explained to be raised to increase type I allergic reactions.

Such patients are predicted to show further aggravated inflammatory symptoms due to infections with Candida, etc. and allergy. Furthermore, administration of steroid to such patients is likely to lead to a further decrease in the Th1 cell function, which already is reduced, due to the suppressing effect of steroid. Thus, the decrease in Th1 cells may be one of the causes of poor steroid responsiveness. Therefore, the gene whose expression level changes in response to steroid responsiveness is expected to be useful as an indicator of the decrease in Th1 cells. Patients having allergic diseases caused by the decrease of Th1 cells not only are poor responder to steroids, but also steroid treatments may involve the risk to be the causative of exacerbation of symptoms in such patients. Therefore, the gene that serves as an indicator for the balance between Th1 and Th2 cells prior to steroid administration is useful.

Alternatively, the testing method of the present invention can be applied to the control of dosage of steroid drugs for patients who already are under continuous steroid therapy. The test based on the present invention can be performed on a patient who clearly showed therapeutic effect by steroid at the beginning of the treatment to predict the development of tendency to poor steroid responsiveness by measuring the decrease in the expression level of the above-described indicator gene. In such a state, no therapeutic effect can be expected with even an increased dosage of steroid. Thus, other therapeutic methods should be considered as much as possible.

Furthermore, the present invention relates to the use of a transgenic, non-human animal whose expression level of the indicator gene in mononuclear cells has been reduced as a poor steroid responsive, allergic disease model animal. The poor steroid responsive, allergic disease model animals are useful for revealing in vivo changes in patients having poor steroid responsive atopic dermatitis. Furthermore, the poor steroid responsive allergic disease model animals based on the present invention are useful in assessing therapeutic methods for poor steroid responsive, allergic atopic dermatitis.

The decrease in the expression levels of the aforementioned indicator gene in mononuclear cells in patients with poor steroid-responsive, allergic disorders was demonstrated by the present invention. Therefore, animals wherein the expression levels of the indicator gene or a gene functionally equivalent thereto in mononuclear cells are artificially suppressed can be used as model animals for poor steroid responsive diseases. Herein, the decrease (or increase) in the expression level in mononuclear cells includes the decrease (or increase) in the expression level of the indicator gene in the whole blood cells. Specifically, the decrease (or increase) in the expression level of the above-described indicator gene includes not only that merely in the mononuclear cell but also that in the whole blood cells and systemic decrease (or increase) of the indicator gene.

In the present invention, a functionally equivalent gene refers to a gene encoding a protein having a similar activity to that demonstrated in the protein encoded by the indicator gene. A typical functionally equivalent gene includes the counterpart of the indicator gene inherent in the species of the transgenic animal. The model animals of poor steroid responsive diseases according to the present invention are particularly useful as model animals of poor steroid responsive allergic diseases.

A gene whose expression level decreases in patients of poor steroid responsive allergic diseases can be referred to as a gene essential for the response to steroid drugs. In other words, the stimulation by steroid drugs is considered to be exerted as an anti-allergic action via the expression of the indicator gene. Therefore, a gene whose expression level is reduced in patients of poor steroid responsive allergic disorders compared to steroid responsive allergic diseases can be referred to a gene that plays an important role in the response to steroid drugs in mononuclear cells. Thus, a drug that promotes the expression of this gene or potentiates the activity of the gene is expected to have an action removing the essential cause of poor steroid responsiveness in steroid therapy for allergies. Moreover, supplementary administration of proteins encoded by these indicator genes enables to achieve the treatment effects of steroid therapy. Supplementation of proteins can also be performed, in addition to the administration of the protein itself, by introducing a vector expressing the indicator gene to patients using gene therapy techniques.

As described above, a gene whose expression level decreases in mononuclear cells of allergic patients with poor steroid responsiveness has important meanings. Therefore, assessment of the role of the gene and efficacy of drugs targeting the gene using, as poor steroid responsive model animals, transgenic animals which can be obtained by reducing the expression level of the gene is highly significant.

Moreover, the poor steroid responsive model animal according to the present invention is useful in the elucidation of steroid response mechanisms and further in testing safety of screened compounds. The model animal for poor steroid responsive disorders according to the present invention is particularly useful as a model animal for poor steroid responsive allergic diseases.

Herein, the phrase “decrease in the expression level” refers to either a state wherein the transcription of the indicator gene inherent in the host or a gene functionally equivalent thereto, and translation of the gene to protein are suppressed. Alternatively, it refers to a state with promoted degradation of proteins, translation products of the gene. The expression level of a gene can be confirmed, for example, by quantitative PCR as shown in Examples. Moreover, the activity of a protein, a translational product, can be confirmed by a comparison to that in the normal state.

Examples of typical transgenic animals include animals having the indicator gene or a gene functionally equivalent thereto knocked out, and those having the indicator gene substituted (knocked in) with another gene. Furthermore, transgenic animals transfected with anti-sense DNA, DNA encoding the ribozyme, or DNA functioning as a decoy nucleic acid, etc. against the indicator gene or a gene functionally equivalent thereto can also be used as a transgenic animal according to the present invention. In addition, animals wherein, for example, mutation has been introduced into the coding region of the indicator gene or a gene functionally equivalent thereto to suppress their activities or modify the amino acid sequence of the protein encoded by the gene into a readily degradable protein are also included as the transgenic animals of this invention. Examples of mutation in the amino acid sequence are substitution, deletion, insertion and addition of amino acid(s). In addition, by mutagenizing the transcriptional regulatory region of the gene, the expression itself of the indicator gene of this invention can be controlled.

Methods for obtaining transgenic animals with a particular gene as a target are known. Specifically, a transgenic animal can be obtained by a method where the gene and ovum are mixed and treated with calcium phosphate; a method where the gene is introduced directly into the nucleus of oocyte in pronuclei with a micropipette under a phase contrast microscope (microinjection method, U.S. Pat. No. 4,873,191); or a method where embryonic stem cells (ES cells) are used. Furthermore, there have been developed a method for infecting ovum with a gene-inserted retrovirus vector, a sperm vector method for transducing a gene into ovum via sperm, or such. Sperm vector method is a gene recombination technique for introducing a foreign gene by fertilizing ovum with sperm after a foreign gene has been incorporated into sperm by the adhesion or electroporation method, and so on (M. Lavitranoet et al., Cell, 57: 717, 1989).

Transgenic animals used as poor steroid responsive model animals of the present invention can be produced using all the vertebrates except for humans. More specifically, transgenic animals having various transgene and showing modified gene expression levels are produced using vertebrates such as mice, rats, rabbits, miniature pigs, goats, sheep, monkeys and cattle.

Furthermore, the present invention relates to a method of screening for a compound to raise steroid responsiveness. According to this invention, the expression level of the indicator gene is significantly lowered in mononuclear cells of patients with poor steroid-responsive allergic diseases. Therefore, compounds enhancing steroid responsiveness can be obtained by selecting compounds elevating the expression level of the gene. The screening method of this invention is particularly preferable for screening for candidate compounds useful in improving steroid responsiveness in patients of poor steroid responsive allergic diseases. Compounds elevating the expression level of the gene herein indicate those having inductive functions on any of the steps of transcription and translation of the gene as well as the expression of the activity of the translated protein.

A method of screening for a compound to raise steroid responsiveness of the present invention can be performed either in vivo or in vitro. This screening can be conducted, for example, according to the following steps. The indicator gene in the screening method of this invention includes, in addition to the indicator gene mentioned above, any genes functionally equivalent thereto. The steps are:

(1) administering a candidate compound to a test animal;

(2) measuring the expression level of the above-described indicator gene in a biological specimen of the test animal; and

(3) selecting a compound elevating the expression level of the indicator gene compared to that in the control administered with no candidate compound.

According to the screening method of this invention, the CYP1B1 gene or a gene functionally equivalent thereto can be used as an indicator gene. The phrase “functionally equivalent gene” herein refers to a gene encoding a protein having a similar activity to that demonstrated in the protein encoded by the indicator gene. A typical functionally equivalent gene includes a counterpart of the indictor gene inherent in the particular animal species of the test animal.

Test animals of the screening method of this invention include, for example, allergic disease model animals known in the art. For example, a spontaneous dermatitis model using NC/Nga mouse has been reported as a model closely similar to human atopic dermatitis. Administration of mite antigen (5 μg/ear) 8 times in total at 2 to 3-day intervals to the auricle of this mouse induces symptoms closely resembling human atopic dermatitis after 2 weeks or more. The screening according to the present invention can be performed by administering a candidate compound to the above-described animal to monitor changes in the expression level of the indicator gene of this invention.

Thus, the effect of a drug candidate compound on the expression level of the indicator gene can be assess by administering the compound to a test animal and monitoring its action on the expression of the indicator gene in a biological specimen from the test animal. The changes in the expression level of the indicator gene in the biological specimen of the test animal can be monitored by a similar technique to the above-described test method of this invention. Furthermore, the screening for drug candidate compounds can be achieved by selecting drug candidate compounds enhancing the expression level of the indicator gene based on this detection result.

More specifically, the screening according to the present invention can be carried out by collecting a biological specimen from a test animal to compare the expression level of the aforementioned indicator gene to that in the specimen from a control animal administered with no candidate compound. The biological specimens that can be used include lymphocytes and hepatocytes. Preferable biological specimens in the screening method according to this invention are peripheral blood mononuclear cells. Methods for collecting and preparing such biological specimens are known in the art.

The screening enables selection of drugs associated with the expression of the indicator gene in various modes of actions. Specifically, drug candidate compounds having, for example, following actions can be discovered:

(1) activation of the signal transduction pathway causing expression of the indicator gene;

(2) elevation of the transcriptional activity of the indicator gene;

(3) stabilization of the transcripts of the indicator gene or inhibition of decomposition of the transcript, and so on.

Moreover, an in vitro screening method includes, for example, a method comprising contact of a candidate compound with a cell expressing the indicator gene and selection of the compound that elevates the expression level of the gene. The screening can be conducted, for example, according to the steps as described below:

(1) contacting a cell expressing the indicator gene with a candidate compound;

(2) measuring the expression level of the indicator gene; and

(3) selecting a compound elevating the expression level of the indicator gene compared to that in control cells that have not been contacted with the candidate compound.

In this invention, cells expressing the indicator gene can be obtained by inserting the indicator gene into an appropriate expression vector and then transfecting suitable host cells with the vector. Any vectors and host cells may be used as long as they are capable of expressing the gene of this invention. Examples of host cells in the host-vector system are Escherichia coli cells, yeast cells, insect cells and animal cells, and available vectors usable for each can be selected.

Vectors may be transfected into the host by biological methods, physical methods, chemical methods, etc. Examples of the biological methods include methods using virus vectors; methods using specific receptors; and the cell-fusion method (HVJ (Sendai virus) method, the polyethylene glycol (PEG) method, the electric cell fusion method and microcell fusion method (chromosome transfer)). Examples of the physical methods include the microinjection method, the electroporation method and the method using gene particle gun. The chemical methods are exemplified by the calcium phosphate precipitation method, the liposome method, the DEAE-dextran method, the protoplast method, the erythrocyte ghost method, the erythrocyte membrane ghost method and the microcapsule method.

In the screening method of this invention, as cells expressing the indicator gene, peripheral blood leucocytes and cell lines derived therefrom can be used. Mononuclear cells and immature neutrophils can be mentioned as the leucocytes. Among them, lymphoid cell lines are preferable for the screening method of this invention.

According to the screening method of the present invention, first, a candidate compound is added to the above-described cell line. Then, the expression level of the indicator gene in the cell line is measured to select a compound that elevates the expression level of the gene.

In the screening method of this invention, the expression level of the indicator gene can be compared not only based on the expression level of the protein encoded by the gene but also by detecting mRNAs corresponding to the gene. To compare the expression level by mRNA, the step of preparing mRNA samples as described above is carried out in place of the step for preparing a protein sample. mRNA and protein can be detected by performing known methods as mentioned above.

Furthermore, based on the disclosure of this invention, transcriptional regulatory region of the indicator gene of this invention can be obtained to construct a reporter assay system. The phrase “reporter assay system” refers to an assay system for screening a transcriptional regulatory factor that acts on the transcriptional regulatory region using the expression level of a reporter gene that is located downstream of the transcriptional regulatory region as an indicator.

Specifically, this invention relates to a method of screening for therapeutic agents to raise steroid responsiveness, which comprises the steps of:

(1) contacting a candidate compound with a cell transfected with a vector containing the transcriptional regulatory region of an indicator gene and a reporter gene that is expressed under the control of this transcriptional regulatory region;

(2) measuring the activity of the above-described reporter gene; and

(3) selecting a compound that elevates the expression level of the reporter gene compared to that in a control cell which has not been contacted with the candidate compound wherein the indicator gene is the CYP1B1 gene or a gene functionally equivalent thereto.

The transcriptional regulatory region is exemplified by the promoter and enhancer, as well as CAAT box, TATA box and the like which are usually found in a promoter region. Reporter genes such as the chloramphenicol acetyltransferase (CAT) gene, the luciferase gene, growth hormone genes and the like can be utilized in the present invention.

Alternatively, a transcriptional regulatory region of the indicator gene of the present invention can be obtained as follows. Specifically, first, based on the nucleotide sequence of the indicator gene disclosed in this invention, a human genomic DNA library, such as BAC library and YAC library, is screened by a method using PCR or hybridization to obtain a genomic DNA clone containing the sequence of the cDNA. Based on the sequence of the obtained genomic DNA, the transcriptional regulatory region of a cDNA disclosed in this invention is predicted and obtained. The obtained transcriptional regulatory region is cloned upstream of a reporter gene to prepare a reporter construct. The obtained reporter construct is introduced into a cultured cell strain to prepare a transformant for screening. By contacting a candidate compound with this transformant and selecting the compound that induces the expression of the reporter gene in comparison to a control that has not been contacted with the candidate compound, it is possible to perform the screening according to this invention.

As an in vitro screening method according to this invention, a method based on the activity of an indicator protein can be utilized. That is, the present invention relates to a method of screening for therapeutic agents to raise steroid responsiveness, which comprises the steps of:

(1) contacting a candidate compound with a protein encoded by an indicator gene;

(2) measuring the activity of the protein; and

(3) selecting a compound that increases the activity of the protein compared to a control protein that has not been contacted with the candidate compound, wherein the indicator gene is a gene functionally equivalent to the CYP1B1 gene.

The activity of CYP1B1, the indicator protein of this invention, is already described above. Using this activity as an indicator, compounds having the activity to elevate the activity of the protein can be screened. The compounds that can be obtained by the method, promote the activity of the CYP1B1 protein. As a result, it is possible to control poor steroid responsive allergic diseases through the activation of the indicator protein whose expression in mononuclear cells is reduced.

Test candidate compounds used in these screening methods include, in addition to compound preparation libraries synthesized by combinatorial chemistry, mixtures of multiple compounds such as extracts from animal or plant tissues, or microbial cultures and their purified preparations.

The polynucleotide, antibody, cell line or model animal, which are necessary for the various methods of screening of this invention, can be combined in advance to produce a kit. More specifically, such a kit may comprise, for example, a cell that expresses the indicator gene and a reagent for measuring the expression level of the indicator gene. As a reagent for measuring the expression level of the indicator gene, for example, an oligonucleotide that has at least 15 nucleotides complementary to the polynucleotide comprising the nucleotide sequence of at least one indicator gene or to the complementary strand thereof is used. Alternatively, an antibody that recognizes a peptide comprising the amino acid sequence of at least one indicator protein may be used as a reagent. In these kits may be packaged a substrate compound used for the detection of the indicator, medium and a vessel for cell culturing, positive and negative standard samples, and furthermore, a manual describing how to use the kit.

Compounds selected by the screening methods of this invention are useful as a drug to raise steroid responsiveness. Furthermore, proteins encoded by the indicator gene of the present invention or genes functionally equivalent thereto are useful as a drug to raise steroid responsiveness. A drug to raise steroid responsiveness of this invention can be formulated by including a compound selected by the above-described screening methods, or a protein encoded by the indictor gene of this invention or genes functionally equivalent thereto as the effective ingredient, and mixing it with physiologically acceptable carrier, excipient, diluent and the like. For improving steroid responsiveness in patients with disorders for whom the administration of steroid drugs has been selected as a therapeutic method, the drug to raise steroid responsiveness of this invention can be administered orally or parenterally. Disorders for which the drug of this invention is applied include poor steroid responsive allergic diseases. Alternatively, when the compound to be administered consists of a protein, a therapeutic effect can be achieved by introducing a gene encoding the protein into the living body using techniques of gene therapy. Techniques for treating disorders by introducing, into the living body, a gene encoding a protein with a therapeutic effect and expressing the gene in vivo is known in the art.

For oral drugs, any dosage forms including granules, powders, tablets, capsules, solutions, emulsions and suspensions may be selected. Injections are exemplified by subcutaneous, intramuscular and intraperitoneal injections.

Moreover, compounds that can be obtained by the screening methods of this invention include those having the activity to improve and raise steroid responsiveness of patients and which thus are useful as drugs. Such drugs can be formulated as therapeutic agents for poor steroid responsive diseases by combining them with steroids.

Although the dosage may vary depending on the age, sex, body weight, symptoms of a patient, treatment effects, method for administration, treatment duration, type of active ingredient contained in the drug composition, etc., a range of 0.1 to 500 mg, preferably, 0.5 to 20 mg per dose for an adult can be administered. However, the dose changes according to various conditions, and thus in some case a more smaller amount than that mentioned above is sufficient whereas an amount above the above-mentioned range is required in other cases.

All the literatures for prior arts cited in the present specification are herein incorporated by reference.

### BRIEF DESCRIPTION OF THE DRAWINGS

FIG. 1 represents bar graphs showing the results of the measurements on the CYP1B1 gene expression levels in the steroid responder group, poor steroid responder group and normal healthy individuals. The upper graph shows the measured values (copy/ng RNA) in each subject corrected for the β-actin gene. The lower graph represents the results of statistical analysis among respective groups. Herein, V represents a normal healthy subject, R the steroid responder group, and P the poor steroid responder group. Numerals are the reference numbers of respective subjects.

FIG. 2 represents bar graphs showing the results of the measurements on the CYP1B1 gene expression levels in the steroid responder group, poor steroid responder group and normal healthy individuals. The upper graph shows the measured values (copy/ng RNA) in each subject corrected for the GAPDH gene. The lower graph represents the results of statistical analysis among respective groups. Herein, V represents a normal healthy subject, R the steroid responder group, and P the poor steroid responder group. Numerals are the reference numbers of respective subjects.

### BEST MODE FOR CARRYING OUT THE INVENTION

The present invention will be explained in more detail below with reference to examples, but it is not to be construed as being limited thereto.

### EXAMPLE 1

### Selection of Candidate Gene Using DNA Chip

(1) Mononuclear Cells

Heparinized blood samples were withdrawn from 2 normal healthy volunteers (hereinafter referred to as “normal group”), 3 responders to steroid ointment treatment and 3 poor-responders thereto (hereinafter referred to as “steroid responder group” and “poor steroid responder group”, respectively; also both groups collectively referred to as “patient group”). Then the blood samples were subjected to specific gravity centrifugation according to following method for collecting mononuclear cell fractions to culture the fractions.

40-ml of the whole blood (using a heparin anticoagulant at a final concentration of 50 unit/ml) was placed in a centrifuge tube; an equal volume of 3% dextran/0.9% NaCl was added and mixed by gently tumbling the tube several times. The resulting mixture was left standing at room temperature for 30 min. Then, the supernatant (platelet rich plasma) was recovered and centrifuged at 1,200 rpm (revolutions per minute) at room temperature for 5 min. After removing the supernatant, the pellet was suspended in Hank's Balanced Salt Solutions (HBSS, GIBCO BRL) (5 ml), layered on Ficoll-Paque™ PLUS (Amersham Pharmacia Biotech) (5 ml), centrifuged at 1,200 rpm at room temperature for 5 min and further for 30 min raising the rpm to 1,500 at room temperature. The supernatant was removed to recover the intermediate layer. The recovered layer was suspended in PBS and centrifuged at 1,500 rpm at room temperature for 5 min. The supernatant was discarded. The pellet was re-suspended in PBS and centrifuged at 1,500 rpm at room temperature for 5 min. The pellet thus obtained was suspended in RPMI1640 (GIBCO BRL)/10% FCS (SIGMA) (10 ml). 20 μl of the suspension was subjected to cell staining with Trypan Blue Stain 0.4% (GIBCO BRL) to count the cell number. A suspension (1.5×10⁶ cells/ml) in RPMI1640/10% FCS (10 ml) was prepared and cultured at 37° C. in a 5% CO₂ atmosphere for 24 h. Then total RNA was extracted according to following method.

Total RNA was extracted using RNA extraction kit, ISOGEN (Nippon Gene) according to the accompanying direction. The cultured cells were lysed in Isogen (4 M guanidium thiocyanate, 25 mM sodium cyanate, 0.5% Sarcosyl, 0.1 M β-mercaptoethanol, pH 7.0) (3 ml). Suction using a 2.5-ml syringe with a 20 G Cathelin needle was repeated 20 to 30 times. CHCl₃ (0.6 ml, ⅕ volume of Isogen) was added to the extract, mixed for 15 sec using a mixer and the mixture was left standing at room temperature for 2 to 3 min. Then, the mixture was centrifuged at 15,000 rpm, 4° C. for 15 min. The supernatant was transferred into a fresh tube, Ethachinmate (Nippon Gene) (3 μl) and isopropanol (1.5 ml, ½ volume to Isogen) were added thereto, mixed by tumbling and the resulting mixture was left standing at room temperature for 10 min. After the mixture was centrifuged at 15,000 rpm, 4° C. for 15 min, 75% ethanol (3 ml, equal volume to Isogen) was added to the precipitate, and the mixture was centrifuged at 15,000 rpm, 4° C. for 5 min. The precipitate was air-dried or vacuum-dried for 2 to 3 min, and RNase-free DW (10 μl) was added to prepare an RNA solution.

(2) Synthesis of cDNA for DNA Chip

Single-stranded cDNA was prepared by reverse-transcription from the total RNA (2 to 5 μg) using a T7-(dT)₂₄ (Amersham Pharmacia Biotech) as a primer and Superscript II Reverse Transcriptase (Life Technologies) according to the method described in Expression Analysis Technical Manual (Affymetrix). The T7-(dT)₂₄ primer consists of the nucleotide sequence of T7 promoter to which (dT)₂₄ is added. T7-(dT)₂₄ primer (SEQ ID NO: 1):

5′-GGCCAGTGAATTGTAATACGACTCACTATAGGGAGGCGG-(dT)₂₄-3′

Then, according to the Expression Analysis Technical Manual, DNA Ligase, DNA polymerase I and RNase H were added to the above-described single-stranded cDNA to synthesize a double-stranded cDNA. The cDNA was purified by phenol-chloroform extraction, passing through Phase Lock Gels and ethanol precipitation.

Furthermore, using BioArray High Yield RNA Transcription Labeling Kit, biotinylated cRNA was synthesized, purified using an RNeasy Spin column (QIAGEN) and then fragmented by heat treatment.

12.5 μg of cRNA was added to a Hybridization Cocktail according to the Expression Analysis Technical Manual. The resulting mixture was added to a DNA microarray, and subjected to hybridization at 45° C. for 16 h. GeneChip® HuGeneFL (Affymetrix) was used as the DNA chip, which is composed of probes consisting of the nucleotide sequences derived from approximately 5600 kinds of human cDNAs and ESTs.

The DNA chip was washed and then stained by adding Streptavidin Phycoerythrin thereto. After washing, an antibody mixture containing normal goat IgG and biotinylated goat anti-streptavidin IgG antibody was added to the microarray. Furthermore, to enhance the fluorescence intensity, the microarray was restained by adding Streptavidin Phycoerythrin. After washing, the microarray was set on a scanner and analyzed with GeneChip Software.

(3) DNA Chip Analysis

The expressed fluorescence intensities were measured for data analyses using DNA chip analysis software, Suite. First, all of the chips were subjected to Absolute analysis to measure the gene expression level in each of the used samples.

In the analysis of a single chip data, the fluorescence intensities of the perfect match and mismatch of the probe set were compared to determine positive and negative fractions. The results were classified based on the values of Positive Fraction, Log Avg and Pos/Neg into three groups of Absolute Calls: P (present), A (absent) and M (marginal). Definitions of these terms are described below:

Positive Fraction: ratio of Positive pairs;

Log Avg: logarithmic mean of fluorescence intensity ratios between perfect match and mismatch probe cells; and

Pos/Neg: ratio of Positive pair numbers and Negative pair numbers.

Moreover, Average Difference (Avg Diff), i.e., the mean value of the difference in the fluorescence intensity between perfect match and mismatch probe cells was also calculated.

Next, two data were compared. In the comparative experiment, a chip for standard was determined, and Comparison Analysis was performed using the total gene expression level of the standard chip as a reference standard. Comparison Analysis was performed for one steroid responsive patient against 3 poor steroid responsive patients and the result was used as the standard. Genes whose expression levels in the steroid responsive patient used as the standard are high were limited to genes with a fold change value, one of the calculated values in the software, of −3 or less and at the same time to those satisfying either (i) or (ii) as follows:

(i) genes with a gene expression judgment standard (Absolute call) of P (present) in steroid responsive patients; and

(ii) genes with a gene expression judgment standard (Absolute call) of A (absent) or M (marginal) in poor steroid responsive patients, and with an expression judgment standard M (marginal) in steroid responsive patients.

Then, genes with a difference call value of NC (Not change) MD (Marginal Decrease) or D (Decrease) were selected. On the other hand, genes whose expression levels are low were limited to genes with a fold change value of 3 or more, and at the same time satisfying (i) or (ii) as follows:

(i) genes with an Absolute call of P (present) in poor steroid responsive patients; and

(ii) genes with an Absolute calls of A (absent) or M (marginal) in steroid responsive patients, and an expression judgment standard of M (marginal) in poor steroid responsive patients.

Then, genes with a difference call value of NC (Not change) MD (Marginal Decrease) or D (Decrease) were selected. Next, according to a graph using scattered plots of Avg Diff values in the log scale, genes plotted near the origin were omitted.

As for genes selected using an analytical software, Suite, genes selected according to the results of 6 different analyses based on two standard patients were chosen among the genes with a high gene expression level in steroid responsive patients.

Response 1 vs. Poor response 1, poor response 2, poor response 3

Response 2 vs. Poor response 1, poor response 2, poor response 3

The classification of genes selected by GeneChip Comparison Analysis showing similar expression changes in the poor steroid responder group by the above-described 6 different combinations are shown in Table 1. Genes with a change of 3-fold or more, or ⅓ or less from the raw data measured values are shown.

TABLE 1

Poor responder group

| | Increase | Decrease |
|---|---|---|
| Responder group | 4 | 2 |

To correlate the results with ABI7700, the expression levels were respectively corrected for the β-actin gene based on Avg Diff values of Absolute analysis to finally select genes showing interesting changes between the steroid responder and poor steroid responder groups.

As a result, the CYP1B1 gene was selected as a gene showing a decrease of ⅓ or less in the expression level in the poor steroid responder group. The expression level of the CYP1B1 gene decreases in poor steroid responsive patients with allergic dermatitis, and the gene is closely associated with poor steroid responsive allergic dermatitis.

### EXAMPLE 2

### Expression Level of CYP1B1 Gene in Peripheral Blood Mononuclear Cells and Atopic Dermatitis

For quantitative confirmation of the expression level of the CYP1B1 gene selected in Example 1, quantitative PCR by ABI 7700 was further performed with PBMC as a specimen.

The changes in the expression of the CYP1B1 gene, which had been considered associated with the pathophysiology of steroid responsive allergic diseases were analyzed in mononuclear cells isolated from peripheral blood (peripheral blood mononuclear cells, PBMC) of atopic dermatitis patients and normal healthy subjects.

7 normal healthy volunteers, 5 responders to steroid ointment therapy and 6 poor-responders thereto were used as subjects. Isolation and culture of PBMC (peripheral blood mononuclear cell) and extraction of RNA for quantification of the gene expression level in this Example were carried out according to the methods as described in Example 1 (1). Operation of reverse transcription reaction and quantitative PCR method were performed as described below.

(1) DNase Treatment of Total RNA

The total RNA solution (20 μg), 10× DNase Buffer (5 μl) (Nippon Gene), RNase inhibitor (Amersham Pharmacia Biotech) (25 units) and DNase I (Nippon Gene) (1 unit) were mixed and DNase and RNase-free water was added to a final volume of 50 μl. After incubation at 37° C. for 15 min, water-saturated phenol (pH 8.0) and CHCl₃ (25 μl each) were added to the mixture and mixed by tumbling. After centrifuging at 15,000 rpm at room temperature for 15 min, 3 M sodium acetate (pH 5.2) (5 μl), ethanol (125 μl) and Ethachinmate (1 μl) were added to the supernatant, and the resulting mixture was left standing at −20° C. for 15 min. After centrifuging at 15,000 rpm at 4° C. for 15 min, 80% ethanol (125 μl) was added to the precipitate, and the mixture was centrifuged at 15,000 rpm at 4° C. for 5 min. The precipitate was air-dried or vacuum-dried for 2 to 3 min, and dissolved in RNase-free distilled water (10 μl) to measure its absorbance as an RNA solution.

(2) Reverse Transcription Reaction

The RNA solution (1 to 5 μg), Oligo (dT)₁₂₋₁₈ primer (GIBCO BRL) (500 ng) and BSA (1 μg) were mixed and adjusted to a final volume of 12 μl with sterilized distilled water. The mixture was left standing at 70° C. for 10 min, and then cooled on ice. 5× First Strand Buffer (GIBCO BRL) (4 μl), 1 M DTT (2 μl) and 10 mM dNTPs (1 μl) (N=G, A, T, C) were added to the mixture and mixed. After heating the mixture at 42° C. for 2 min, SuperScriptII (GIBCO BRL) (200 units) was added thereto, and the mixture was reacted at 42° C. for 50 min. Then, the mixture was treated at 70° C. for 15 min to inactivate the reverse transcriptase. RNase H (GIBCO BRL) (2 units) was added thereto and incubated at 37° C. for 20 min. Sterilized distilled water was added to the mixture to prepare a cDNA solution of a concentration of 10 ng/μl and the solution was subjected to quantitative PCR.

(3) PCR Amplification of Target Region

10× PCR Buffer (100 mM Tris-HCl, pH 8.3, 500 mM KCl, 15 mM MgCl₂) (5 μl), 2.5 mM dNTPs (4 μl) (N=G, A, T, C), primer F (10 pmol/μl), primer R (10 pmol/μl), cDNA solution (5 ng) and rTaq DNA polymerase (TaKaRa) (1.25 units) were mixed and adjusted to a final volume of 50 μl with sterilized distilled water. The primers had the following nucleotide sequences:

primer F: 5′- TTA TGA AGC CAT GCG CTT CT -3′/SEQ ID NO: 2; and

primer R: 5′- AAG ACA GAG GTG TTG GCA GTG -3′/SEQ ID NO: 3.

After the mixture was left standing at 95° C. for 10 min, 40 cycles of “95° C. for 15 s and 60° C. for 1 min” were carried out. Then, electrophoresis on 3% agarose gel (Agarose-1000, GIBCO-BRL)/5 μg/ml ethidium bromide in electrophoresis buffer solution 1× TAE (50× TAE contains Tris base (242 g), glacial acetic acid (57.1 ml) and 50 mM EDTA (pH 8.0) in 1 liter) at 100 V for 30 min was conducted. Then, the gel was scanned under an UV lamp to observe the band for a PCR product of 75 bp.

(4) Excision of DNA Fragments

The PCR product of interest was excised from the gel using QIAEX II Agarose Gel Extraction kit (QIAGEN) according to the accompanying manual. After the isolation of the PCR products by electrophoresis on a 3% agarose gel, the fragment of interest was excised under a long wavelength (316 nm) UV. The gel was macerated using a razor, and transferred into a 1.5-ml tube (˜250 mg gel). 6 volumes of Buffer QXl (300 μl for excised gel 50 mg) and QIAEX II glass bead (10 μl) was added and the mixture was thoroughly mixed for 30 s using a vortex mixer. The resulting mixture was heated at 50° C. for 10 min with mixing at several minutes intervals until the mixture became yellow. When the color of the mixture was orange or purple, 3 M sodium acetate (pH 5.0) (10 μl) was added. After centrifugation at 12,000 rpm at room temperature for 30 s, Buffer QXl (500 μl) was added to the precipitate, thoroughly vortexed, and the mixture was centrifuged at room temperature and 12,000 rpm for 30 s. Then, PE solution (500 μl) was added to the precipitate, and centrifuged at room temperature at 12,000 rpm for 30 sec (process (A)). The process (A) was repeated twice. Then, the supernatant was discarded and the precipitate was dried until it became white. Sterilized distilled water (20 μl) was added to the precipitate, and after leaving standing for 5 min, the mixture was centrifuged at room temperature at 12,000 rpm for 30 sec to recover the supernatant (process (B)). After repeating process (B) twice, the supernatant was subjected to agarose gel electrophoresis to confirm the extraction of the PCR product.

(5) TA Cloning of PCR Product

Cloning of the purified PCR product was conducted using a pGEM^{R}-T Easy Vector System I (Promega) according to the accompanying manual. 2× Rapid Ligation Buffer (5 μl), PGEM^{R}-T Easy Vector (50 ng/μl) (1 μl), the purified PCR product (3 μl) and T4 DNA Ligase (3 Weiss units/μl) (1 μl) were mixed and left standing at room temperature for 1 h (or at 16° C. overnight). Ligation reaction solution (2 μl) was added to Competent Cells DH5α (GIBCO BRL) (50 μl), and the resulting mixture was left on ice for 20 min. Then, heat shock treatment at 42° C. for 45 to 50 sec was conducted, and the treated mixture was left standing on ice for 2 min. SOC medium (GIBCO BRL) (950 μl) was added to the cells and mixed at 37° C. for 1 to 1.5 h at 150 rpm. The cell culture (100 μl) was plated on LB/amp/IPTG/X-gal and left standing at 37° C. overnight.

(6) Plasmid DNA Extraction

The subcloned plasmid DNA was extracted using Wizard Plus SV Minipreps DNA Purification System (Promega) according to the accompanying manual. First, white colonies were picked up, cultured in ampicillin (100 μg/ml)-LB medium (1 to 5 ml) at 37° C. overnight, and then centrifuged at 3,000 rpm for 6 min. Resuspended solution (250 μl) was added to suspend the precipitate; Lysis solution (250 μl) was added thereto and mixed 4 times by tumbling. Alkaline protease (10 μl) was added thereto, mixed 4 times by tumbling and the mixture was left standing at room temperature for 5 min. Neutralization solution (350 μl) was added to the mixture, mixed 4 times by tumbling, and centrifuged at room temperature at 14,000 rpm for 10 min. Then, the supernatant was transferred on a column included in the kit by decantation and centrifuged at room temperature at 14,000 rpm for 10 min. 700 μl of wash solution was added to the column portion (the follow-through fraction was discarded), and the mixture was centrifuged at room temperature at 14,000 rpm for 1 min. Then, 250 μl of the wash solution was added to the column portion (the follow-through fraction was discarded), and the mixture was centrifuged at room temperature at 14,000 rpm for 2 min. The column portion was transferred into a fresh tube, sterilized distilled water (20 μl) was added thereto, and the mixture was centrifuged at room temperature at 14,000 rpm for 1 min. The obtained solution was used as a plasmid DNA preparation and its concentration was determined by absorbance measurement.

(7) Sequence Reaction

Sequence reaction for confirming whether the subcloned plasmid DNA contains the DNA sequence of interest or not was performed using Thermo Sequinase II dye terminator (Amersham Pharmacia Biotech) according to the accompanying manual. First, M13 primer (3 pmol), the DNA solution (200 to 300 ng) and TSII Reagent Mix (2 μl) were mixed and adjusted to a final volume of 10 μl with sterilized distilled water. After leaving standing at 96° C. for 1 min, 30 cycles (96° C. for 30 sec, 50° C. for 15 sec, and 60° C. for 1 min as one cycle) were performed and then the temperature was lowered to 4° C. Then, 1.5 M sodium acetate/250 mM EDTA (1 μl) was added to the reaction solution and vortexed. Isopropanol (20 μl) was added, thoroughly mixed, and the mixture was left standing at room temperature for 10 min. After centrifugation at 12,000 rpm for 20 min, 70% ethanol (150 μl) was added to the precipitate and mixed. The mixture was then centrifuged at 12,000 rpm for 5 min, and the precipitate was air-dried or vacuum-dried for 2 to 3 min. Next, after the addition of loading dye (1.5 μl) to the dried precipitate, the mixture was subjected to a heat treatment at 95° C. for 2 min, and then cooled on ice. The whole reaction product was applied on a LongRanger gel [LongRanger (5 ml), urea (15 g), 10× TBE (5 ml), 10% APS (250 μl) and TEMED (35 μl), adjusted to a final volume of 50 ml with sterilized distilled water] set on ABI377 DNA sequencer (Applied Biosystems) to start electrophoresis. After confirming the PCR product to contain the objective DNA sequence, the product was used as the standard sample.

(8) Quantitative PCR

Quantification of the gene expression level was carried out by real-time PCR using ABI PRISM 7700 System with TaqMan probe according to the accompanying manual. TaqMan 1000 Reaction PCR Core reagents (Applied Biosystems) were used according to the accompanying manual as the reaction reagent. At least 5 gradients between 10⁷ to 10³ copies of the concentration gradient were prepared as the standard samples for plotting a calibration curve. The “n” number per one sample was set as at least 2. 10× Buffer A (5 μl), 25 mM MgCl₂ (7 μl), 10 mM dNTPs (1 μl each) (N=G, A, T, C), AmpTaqGold (1.25 units), UNG (0.5 units), primer F (10 pmol), primer R (10 pmol), cDNA solution (5 ng) and TaqMan Probe (5 pmol) were mixed and adjusted to a final volume of 50 μl with sterilized distilled water. As the primers for amplification, the same as in (3) (SEQ ID NOs: 2 and 3) were used and the probe had the following nucleotide sequence:

TaqMan probe: 5′- (FAM) CAG CTT TGT GCC TGT CAC TAT TCC TCA TG -3′ (TAMRA)/SEQ ID NO: 4

FAM: 6-carboxyfluorescein

TAMRA: 6-carboxy-tetramethylrhodamine

After leaving the above reaction mixture standing at 50° C. for 2 min and then at 95° C. for 10 min, 50 cycles (95° C. for 15 sec and 60° C. for 1 min as one cycle) were performed. A calibration curve was automatically plotted from the Ct (threshold cycles) value of PCR amplification curve plotted against the logarithm of relative initial concentrations of the standard sample. Then, based on the calibration curve, relative initial concentrations of cDNA in unknown samples were calculated.

In order to correct the difference in the cDNA concentrations among samples, a similar quantitative analyses were carried out for the β-actin gene and glyceraldehyde-3-phosphate dehydrogenase (GAPDH) gene as the internal standard for correction to calculate the copy number of the target gene based on their copy numbers.

As the primers and probes for the measurement of β-actin and GAPDH genes, those included in TaqMan β-actin Control Reagents (Applied Biosystems) were utilized. Their nucleotide sequences were as follows:

| β-actin forward primer | |
|---|---|
| TCA CCC ACA CTG TGC CCA TCT ACG A; | (SEQ ID NO: 5) |
| β-actin reverse primer | |
| CAG CGG AAC CGC TCA TTG CCA ATG G; | (SEQ ID NO: 6) |
| β-actin TaqMan probe | |
| (FAM)ATGCCC-T(TAMRA)-CCCCCATGCCATC | (SEQ ID NO: 7) |
| CTGCGTp-3′; | |
| GAPDH forward primer | |
| GAAGGTGAAGGTCGGAGT; | (SEQ ID NO: 8) |
| GAPDH reverse primer | |
| GAAGATGGTGATGGGATTTC; and | (SEQ ID NO: 9) |
| GAPDH TaqMan probe | |
| (FAM)CAAGCTTCCCGTTCTCAGCC(TAMRA)- | (SEQ ID NO: 10) |
| 3′. | |

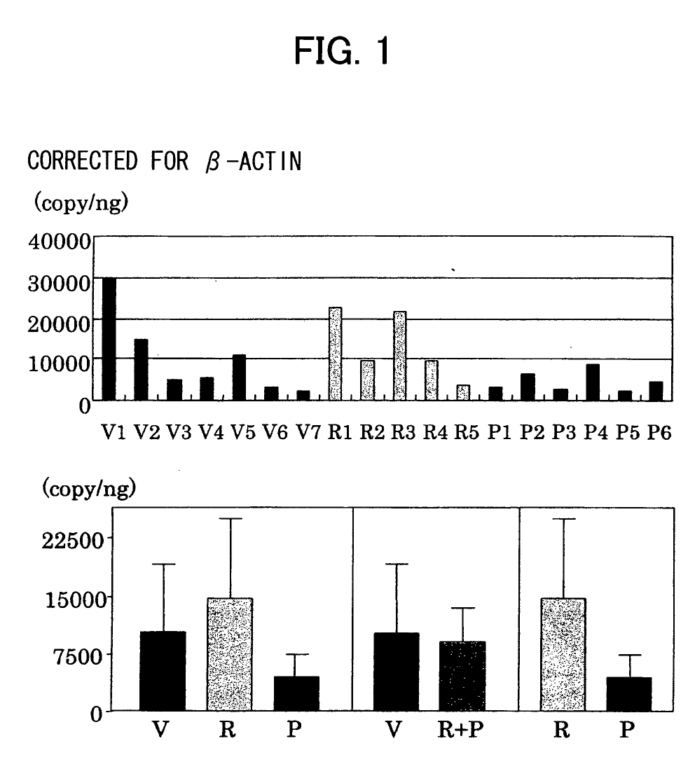

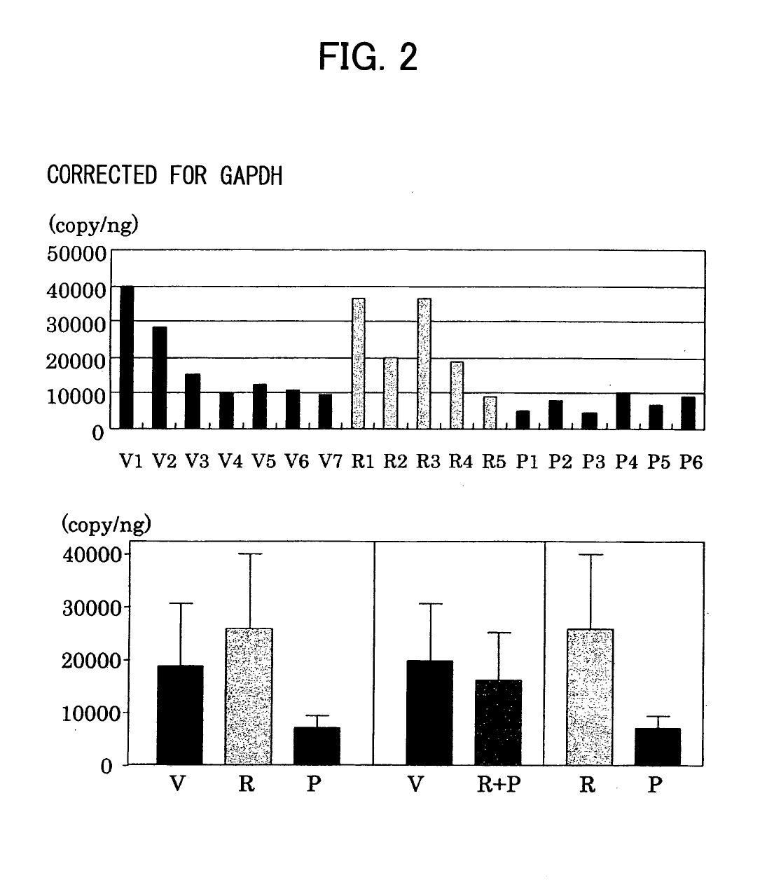

Measurement results are shown in Table 2. Furthermore, based on the measured values, the expression level (copy/ng RNA) of the CYP1B1 gene corrected for β-actin are shown in FIG. 1 (upper panel), and that corrected for GAPDH in FIG. 2 (upper panel).

TABLE 2

| | mRNA expression level (copy/ng) | | |
|---|---|---|---|
| | | Corrected for | Corrected for |
| Type | Raw data | β-actin | GAPDH |
| V1 | 15891 | 29718 | 39760 |
| V2 | 20155 | 14600 | 28233 |
| V3 | 4077 | 5031 | 15495 |
| V4 | 2192 | 5574 | 10330 |
| V5 | 13913 | 10884 | 12782 |
| V6 | 3542 | 3261 | 10548 |
| V7 | 2305 | 2357 | 9670 |
| R1 | 15207 | 22471 | 36416 |
| R2 | 5347 | 9783 | 20145 |
| R3 | 14619 | 21611 | 36474 |
| R4 | 3262 | 9837 | 18915 |
| R5 | 1692 | 3514 | 9240 |
| P1 | 1130 | 3330 | 5186 |
| P2 | 2167 | 6379 | 7697 |
| P3 | 660 | 2835 | 4469 |
| P4 | 2075 | 8795 | 10483 |
| P5 | 2779 | 2468 | 6561 |
| P6 | 1805 | 4807 | 8882 |

| | V (n = 7) | R (n = 5) | P (n = 6) |
|---|---|---|---|
| Raw data | 8868 ± 7540 | 8025 ± 6423 | 1769 ± 763 |
| Corrected for β-actin | 10204 ± 9648 | 13443 ± 8264 | 4769 ± 2446 |
| Corrected for GAPDH | 18117 ± 11514 | 24238 ± 11917 | 7213 ± 2271 |

(9) Statistical Analysis

The statistical analysis of 7 healthy normal volunteers (V group), 5 responders to steroid ointment treatment (R group) and 6 poor-responders to said treatment (P group) were performed by the Fisher's analysis of variance (ANOVA) and the Kruskal-Walli test for the comparisons among 3 groups, and the comparisons between 2 groups, either between normal (V) and patient (R+P) groups, or between responder (R) and poor-responder (P) groups were performed by the Fisher's analysis of variance and the Mann-Whitney test. Analytical results are shown in Table 3, FIG. 1 (lower panel) and FIG. 2 (lower panel).

TABLE 3

| | Comparison between groups P/R | | |
|---|---|---|---|
| | | ANOVA | Mann-Whitny |
| | Difference | p value | p value |
| Raw data | R > P | 0.0404 | 0.0446 |
| Corrected for | R > P | 0.0357 | 0.0285 |
| β-actin | | | |
| Corrected for | R > P | 0.0071 | 0.0106 |
| GAPDH | | | |

As judged from the data obtained by the quantitative PCR, the expression level of the CYP1B1 gene selected in Example 1 in mononuclear cells decreased to ½-fold or less to the control value in steroid poor-responder group. Based on these results, the decrease in the expression level of the CYP1B1 gene in mononuclear cells was suggested to serve as an indicator for poor responsiveness to steroid of allergic disease patients.

### Industrial Applicability

According to the present invention, a gene with a decreased expression level in mononuclear cells in a poor steroid responder group was revealed. The gene whose expression level in mononuclear cells is lowered in the poor steroid responder group serve as an indicator for poor responsiveness to steroid of allergic dermatitis patients. Furthermore, the gene of the present invention is expected to be useful as an indicator for the decrease in Th1 cells.

The decrease in the expression level of the indicator gene of the present invention is associated with the responsiveness to steroid drugs. Thus, elevation of the expression level of the gene serves as a target of therapeutic strategy for disorders for which steroid administration is selected as a treatment. Furthermore, the gene is also expected to be useful as a novel clinical diagnostic indicator for monitoring the effect of such new therapeutic methods. Allergic diseases are typical examples of such disorders. Alternatively, supplementary administration of a protein encoded by the gene to compensate for the decrease in its expression level may function as a therapeutic method for allergic diseases.

Since the method for testing steroid responsiveness of this invention enables analysis of the expression level of the indicator gene with a biological specimen as a test sample, it is less invasive to patients. Furthermore, gene expression analyses allow highly sensitive measurement of the gene expression in a minute quantity of test samples. Year by year, gene analytical techniques are improved for more high-throughput and price-cutting is in progress. Therefore, the method for testing steroid responsiveness according to this invention is expected to become an important bedside diagnostic method in the near future. In this regard, the gene associated with steroid responsiveness is highly valuable in diagnosis.

1. A method for testing steroid responsiveness, comprising the steps of:
a) measuring the expression level of the CYP1B1 gene in a biological sample of a test subject; and
b) comparing the measured expression level to that of the gene in a biological sample from a steroid responsive subject.

2. The method according to claim 1, wherein steroid responsiveness in allergic diseases is tested.

3. The method according to claim 2, wherein the allergic disease is atopic dermatitis.

4. The method according to claim 1, wherein the expression level of the gene is measured by PCR.

5. The method according to claim 1, wherein the expression level of the gene is measured by detecting protein encoded by the gene.

6. A reagent for testing steroid responsiveness, said reagent comprising an oligonucleotide comprising at least 15 nucleotides of the nucleotide sequence of the CYP1B1 gene or to the complementary strand thereof.

7. A reagent for testing steroid responsiveness, said reagent comprising an antibody recognizing peptides comprising the amino acid sequence of the CYP1B1 protein.

8. A method of screening for a compound that raises steroid responsiveness, comprising the steps of:
(1) contacting a candidate compound with a cell that expresses the CYP1B1 gene and/or a gene functionally equivalent thereto;
(2) measuring the expression level of the gene; and
(3) selecting a compound that elevates the expression level of the gene compared to that in a control cell that has not been contacted with the candidate compound.

9. The method according to claim 8, wherein the cell is a mononuclear cell line.

10. A method of screening for a compound that raises steroid responsiveness, comprising the steps of:
(1) administering a candidate compound to a test animal;
(2) measuring the expression intensity of the CYP1B1 gene and/or a gene functionally equivalent thereto in a biological sample from the test animal; and
(3) selecting a compound that elevates the expression level of the gene compared to that of a control animal without administration of the candidate compound.

11. A method of screening for a compound that raises steroid responsiveness, comprising the steps of:
(1) contacting a candidate compound with a cell transfected with a vector comprising the transcriptional regulatory region of the CYP1B1 gene and/or a gene functionally equivalent thereto, and a reporter gene that is expressed under the control of the transcriptional regulatory region;
(2) measuring the activity of the reporter gene; and
(3) selecting a compound that elevates the expression level of the gene compared to that of a control cell that has not been contacted with the candidate compound.

12. A method of screening for a compound that raises steroid responsiveness, comprising the steps of:
(1) contacting a candidate compound with the CYP1B1 protein and/or a protein functionally equivalent thereto;
(2) measuring the activity of the protein; and
(3) selecting a compound that elevates the activity of the protein compared to that of the control protein that has not been contacted with the candidate compound.

13. A pharmaceutical to raise steroid responsiveness, which comprises a compound obtained by the method according to claim 8, as an effective ingredient.

14. A pharmaceutical to raise steroid responsiveness, which comprises the CYP1B1 gene or the CYP1B1 protein as the main ingredient.

15. A therapeutic agent for poor steroid responsive disorders comprising the pharmaceutical to raise steroid responsiveness according to claim 14 in combination with a steroid drug.

16. A kit for screening a candidate compound for a therapeutic agent to raise steroid responsiveness, said kit comprising a polynucleotide comprising at least 15 nucleotides of the nucleotide sequence of the CYP1B1 gene or the complementary strand thereof, and a cell expressing the CYP1B1 gene.

17. A kit for screening a candidate compound for a therapeutic agent to raise steroid responsiveness, said kit comprising an antibody recognizing a peptide comprising the amino acid sequence of the CYP1B1 protein, and a cell expressing the CYP1B1 gene.

18. An animal model of poor steroid responsiveness, wherein said animal is a transgenic non-human vertebrate, whose expression intensity of the CYP1B1 gene and/or a gene functionally equivalent thereto in mononuclear cells is decreased.

19. A pharmaceutical to raise steroid responsiveness, which comprises a compound obtained by the method according to claim 10, as an effective ingredient.

20. A pharmaceutical to raise steroid responsiveness, which comprises a compound obtained by the method according to claim 11, as an effective ingredient.

21. A pharmaceutical to raise steroid responsiveness, which comprises a compound obtained by the method according to claim 12, as an effective ingredient.

22. A therapeutic agent for poor steroid responsive disorders comprising the pharmaceutical to raise steroid responsiveness according to claim 13 in combination with a steroid drug.

23. A therapeutic agent for poor steroid responsive disorders comprising the pharmaceutical to raise steroid responsiveness according to claim 19 in combination with a steroid drug.

24. A therapeutic agent for poor steroid responsive disorders comprising the pharmaceutical to raise steroid responsiveness according to claim 20 in combination with a steroid drug.

25. A therapeutic agent for poor steroid responsive disorders comprising the pharmaceutical to raise steroid responsiveness according to claim 21 in combination with a steroid drug.

26. A method for increasing steroid responsiveness comprising administering to a patient a compound that elevates the expression level of the CYP1B1 gene.


SEQUENCE LISTING (12 sequences)

SEQ ID NO: 1 - DNA, 63 bp, Artificial Sequence
  Synthetic
ggccagtgaattgtaatacgactcactatagggaggcggtttttttttttttttttttttttt

SEQ ID NO: 2 - DNA, 20 bp, Artificial Sequence
  Synthetic
ttatgaagccatgcgcttct

SEQ ID NO: 3 - DNA, 21 bp, Artificial Sequence
  Synthetic
aagacagaggtgttggcagtg

SEQ ID NO: 4 - DNA, 29 bp, Artificial Sequence
  Synthetic
cagctttgtgcctgtcactattcctcatg

SEQ ID NO: 5 - DNA, 25 bp, Artificial Sequence
  Synthetic
tcacccacactgtgcccatctacga

SEQ ID NO: 6 - DNA, 25 bp, Artificial Sequence
  Synthetic
cagcggaaccgctcattgccaatgg

SEQ ID NO: 7 - DNA, 26 bp, Artificial Sequence
  Synthetic
atgccctcccccatgccatcctgcgt

SEQ ID NO: 8 - DNA, 18 bp, Artificial Sequence
  Synthetic
gaaggtgaaggtcggagt

SEQ ID NO: 9 - DNA, 20 bp, Artificial Sequence
  Synthetic
gaagatggtgatgggatttc

SEQ ID NO: 10 - DNA, 20 bp, Artificial Sequence
  Synthetic
caagcttcccgttctcagcc

SEQ ID NO: 11 - DNA, 5102 bp, Homo sapiens
  CDS (347)..(1978)
gcttctgcgactccagttgtgagagccgcaagggcatgggaattgacgccactcaccgacccccagtctcaatctcaacgctgtgaggaaacctcgactttgccaggtccccaagggcagcggggctcggcgagcgaggcacccttctccgtccccatcccaatccaagcgctcctggcactgacgacgccaagagactcgagtgggagttaaagcttccagtgagggcagcaggtgtccaggccgggcctgcgggttcctgttgacgtcttgccctaggcaaaggtcccagttccttctcggagccggctgtcccgcgccactggaaaccgcacctccccgcagcatgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagaagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtgtaatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaagaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 12 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPVKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ